(19) United States
(12) Patent Application Publication
(10) Pub. No.: US 2021/0233641 A1
(43) Pub. Date: Jul. 29, 2021

(54) ANXIETY DETECTION APPARATUS, SYSTEMS, AND METHODS

(71) Applicant: The Texas A&M University System, College Station, TX (US)

(72) Inventors: Farzan Sasangohar, Cypress, TX (US); Anthony D. McDonald, College Station, TX (US)

(21) Appl. No.: 17/050,298

(22) PCT Filed: Apr. 24, 2019
(86) PCT No.: PCT/US2019/028971

§ 371 Date: Oct. 23, 2020

Related U.S. Application Data
(60) Provisional application No. 62/661,939, filed on Apr. 24, 2018.

(51) Int. Cl. G16H 20/70; G16H 10/60; G16H 40/67; G16H 50/20; G06N 20/00; A61B 5/024; A61B 5/16; A61B 5/00

(52) U.S. Cl. CPC G16H 20/70; G16H 10/60; G16H 40/67; G16H 50/20; G06N 20/00; A61B 5/486; A61B 5/165; A61B 5/7267; A61B 5/721; A61B 5/746; A61B 5/7465; A61B 5/02438

(57) ABSTRACT

Patients suffering from a stress- or anxiety-related disorder such as PTSD may utilize wearable/portable sensor and computing technology, e.g., implemented with a smartwatch or smartphone augmented by heartbeat sensors, to continuously monitor their heartbeat data to automatically detect high-stress episodes and take some mitigating action (e.g., alerting the patient, contacting designated persons, or providing stress-reducing exercises and/or content).

### PRIORITY

This application claims the benefit of priority to U.S. Provisional Application Ser. No. 62/661,939, filed Apr. 24, 2018, which is incorporated by reference herein in its entirety.

### TECHNICAL FIELD

The present disclosure relates generally to portable apparatus for managing stress and anxiety disorders.

### BACKGROUND

Post-traumatic stress disorder (PTSD) is a psychiatric disorder that can occur in people who have experienced or witnessed traumatic events such as natural disaster, serious accidents, terrorist attacks, war and combat, or violent crime; the condition is particularly prevalent in combat veterans. Symptoms of PTSD include flashbacks, nightmares, severe anxiety, and uncontrollable thoughts about the event, and, instead of subsiding in time, these symptoms can last for years and be so severe as to interfere with day-to-day functioning. PTSD can be treated with medications, psychotherapy, or a combination of both. Access to treatment, however, may be impeded by geographic, temporal, financial, and cultural barriers. For example, travel distance to facilities, time delays between services, ineligibility for services or high cost of services, and social stigma associated with the condition may all prevent a patient from receiving sufficient care. A related problem is the inability of the treating clinician to monitor and track the occurrence of PTSD episodes in the patient in between visits. Knowing when a patient undergoes a PTSD episode would be useful not only to provide prompt care when the patient needs it, but also to better assess the effectiveness of the overall treatment regimen.

### BRIEF DESCRIPTION OF THE DRAWINGS

Various aspects and example embodiments are described herein with reference to the accompanying drawings, in which:

FIG. 1 is a block diagram providing an overview of anxiety detection systems in accordance with various embodiments;

FIG. 2 illustrates example devices that may be used to implement the system of FIG. 1, in accordance with various embodiments;

FIG. 3 is a flowchart illustrating an anxiety management method as can be performed by the system of FIG. 1, in accordance with various embodiments;

FIG. 4 is a flowchart illustrating a method of creating and training a machine-learning-based algorithm for anxiety detection, in accordance with various embodiments; and

FIG. 5 is a schematic block diagram of an example computing system that may be used for performing anxiety detection and management functionality in accordance with various embodiments.

### DESCRIPTION

Described herein are devices, systems, and methods for managing PTSD or other stress- or anxiety-related (“SAR”) disorders using wearable sensor and real-time processing technology to monitor patients for SAR events and take some type of mitigating action. In particular, in various embodiments, heartbeat data of a patient is acquired and analyzed in real-time, using machine-learning-based classification, to detect when a SAR clinical event (or, herein synonymously, “episode”) occurs. A suitable classification algorithm for that purpose may be trained on a body of continuous heartbeat data and temporally associated self-reports of SAR clinical events obtained from the specific patient or from a larger patient population to output, e.g., the likelihood of a SAR event occurring at any given time based on the heartbeat data at that time. Once trained and deployed for monitoring a particular patient, the algorithm may be further adjusted based on the patient's confirmation or rejection of automatically detected SAR events, allowing the rate of false negatives to be reduced over time. In some embodiments, the effect of heightened physical activity on the heartbeat is accounted for based on accelerometer data indicative of patient movements acquired simultaneously with the heartbeat data, using a classification algorithm trained to discriminate between heartbeat signatures associated with SAR clinical events and heartbeat signatures associated with high physical activity. Detected SAR clinical events may be time-stamped and stored in memory, enabling, e.g., a medical-care provider to later access and evaluate this information.

Detection of a SAR clinical event may, in accordance herewith, trigger an automatic response, such as, without limitation: activation of an alert (e.g., a physical alert perceptible by the patient); automatic communication of the event to a medical-care provider, selected relative or friend, or other person designated by the patient, or prompt to the patient to initiate such communication, e.g., via phone call, text, or email; and/or output (e.g., via screen display or speaker) of content guiding the user through one or more stress-reducing exercises, e.g., involving breathing or muscle-relaxation techniques or meditation. All of these automated responses are examples of “mitigating actions” as broadly understood herein. In accordance with various embodiments, at least some mitigating actions are implemented by the user interface of a portable device carried by the patient. For example, a “mobile app” (i.e., software application running on a cell phone or similar mobile device) may include functionality for providing electronic content to the patient, initiating electronic communications with designated persons, and/or triggering visual or audible indicators or vibration of the device to alert the patient when he undergoes a SAR episode. In some embodiments, the mobile app, or separate software executing on the patient's portable device, also processes the sensor heartbeat data. In other embodiments, the sensor data is sent, via a suitable communications network, to a remote computational facility for processing, and detected SAR events are communicated back to the patient's device.

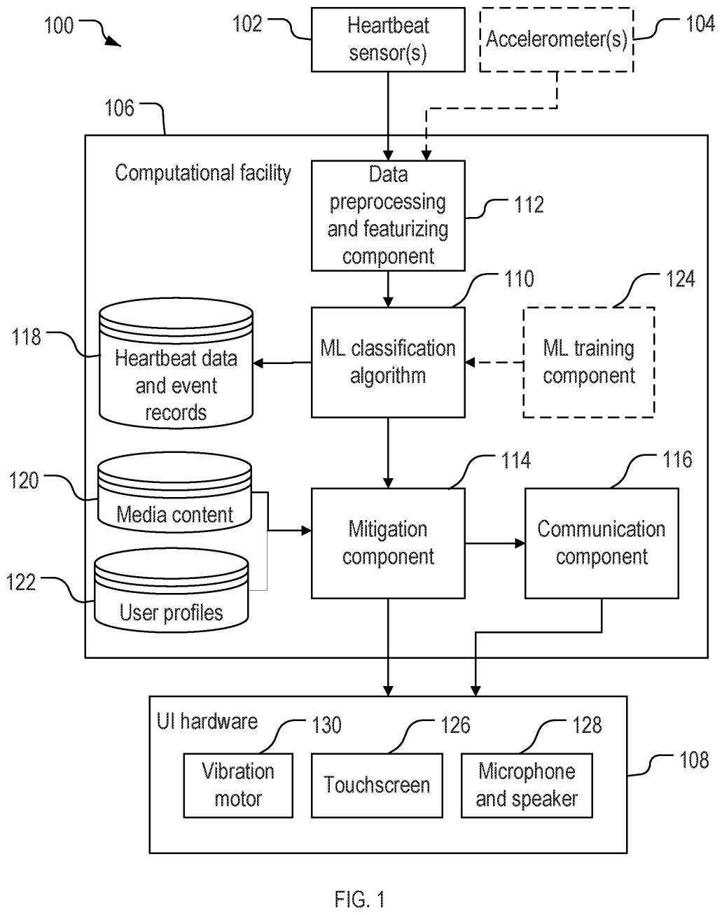

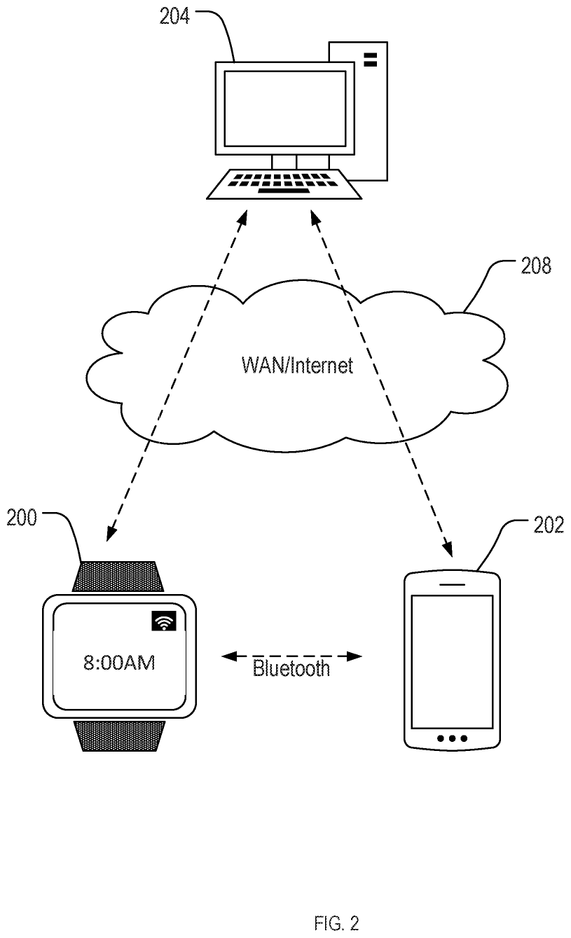

FIG. 1 is a block diagram providing an overview of systems for detecting SAR events (or, briefly, “anxiety detection systems”) in accordance with various embodiments. As depicted, an anxiety detection system 100 may include one or more heartbeat sensors 102, optionally one or more accelerometers 104, a computational facility 106 for processing the sensor data and determining one or more mitigating actions, and user-interface hardware 108. The computational facility 106 may be implemented on a single, stand-alone computing device or, alternatively, be distributed across multiple devices that communicate with one another over one or more suitable communications channels or networks (e.g., Bluetooth for wireless short-distance communications and/or the internet or some other wide area network (WAN) for remote communications); various example embodiments are described in more detail below with reference to FIG. 2. In general, the computational facility 106 includes a suitable combination of hardware and/or software, such as one or more hardware processors and memory storing processor-executable instructions providing the computational functionality described herein, and/or appropriately configured special-purpose processors or circuitry (e.g., a digital signal processor (DSP), application-specific integrated circuit (ASIC), field-programmable gate array (FPG), or the like). The device(s) providing the computational facility 106 may also include one or more device and/or network interfaces for receiving data from the sensors 102, 104, and/or the user-interface hardware 108, and/or for communicating with devices external to the system (e.g., the phone or computer of a designated person contacted in response to a SAR event). Sensors 102, 104 and/or user-interface hardware 108 may, alternatively or additionally, be embedded or otherwise integrated into the device(s).

Heartbeat sensors 102 are generally any sensors capable of measuring, when worn by a patient, the patient's heart rate and/or waveforms associated with the heartbeat. A common type of heartbeat sensor operates on the principle of photo plethysmography (PPG), which involves illuminating the skin (e.g., with a light-emitting diode (LED) and measuring the light reflected or transmitted to a photodiode to detect blood volume changes in the microvascular tissue in and/or immediately underneath the skin. PPG sensors are often worn on the finger (or, alternatively, toe) or on the ear lobes or forehead. They can be found integrated, for example, in Fitbit devices as well as, as of late, in some smartphones. Other types of heartbeat sensors measure electrical signals indicative of cardiac activity. For example, a heart rate monitor often used during physical exercise includes a transmitter attached to a belt worn around the chest and a receiver worn around the wrist. Electrocardiogram (ECG) sensors including a set of (one or more) electrodes placed in contact with the patient's skin may also serve as heartbeat sensors.

Accelerometer(s) 104, e.g., as worn by the patient or integrated into a device carried or worn by the patient (e.g., the device providing the computational facility 106), may be used to measure the linear acceleration along one or more (e.g., three mutually perpendicular) axes to enable tracking the patient's acceleration and velocity, which can provide information about physical activity levels. Accelerometers may, for example, exploit the piezoelectric effect to generate voltage signals that reflect stress on a piezoelectric crystal due to accelerative forces, or sense changes in the capacitance between microstructures affected by accelerative forces. Smartphones are often equipped with such accelerometers to enable determining the orientation of the device. Another example of accelerometers are micro-electromechanical-systems-based (MEMS-based) accelerometers.

The computational facility 106 may include multiple functional components, e.g., implemented as respective groups of processor-executable instructions. Such components may include a machine-learning classification algorithm 110 for detecting SAR signals in heartbeat data; a data preprocessing and featurization component 112 that generates input to the machine-learning algorithm 110 from the raw data received by the sensors 102, 104; a mitigation component 114 that determines and performs a mitigating action in response to detection of a SAR episode, e.g., by issuing alert, initiating communications with designated people, and/or providing guidance or other content; and a communication component 116 that provides one or various means for electronic communications. The communication component 116 may be or include standard programs such as, e.g., an email program, phone/texting application, web browser, etc., which may be tied into the anxiety management system 100 by the mitigation component 114. The computational facility may also store various types of data associated with anxiety management, such as, e.g., time series heartbeat and accelerometer data and/or records of detected SAR events 118; media content 120 (e.g., text, images, audio content, or videos) containing, e.g., guidance for stress-reducing exercises or other information relevant to the disorder, optionally including interactive content; and user-profile information 122, including, e.g., a list of contact persons to whom communications should be directed in the event of a SAR episode. The computational facility 106 may also include a machine-learning training component 124 that trains the algorithm 110 prior to deployment based on an initial corpus of heartbeat data and associated SAR-event indications and/or during deployment based on user feedback. However, the anxiety detection system 100 need not itself include the training component 124 to be functional, but may be equipped with a classification algorithm 110 previously trained on a separate system.

The user-interface hardware 108 may include a screen, e.g., a touchscreen 126 that doubles as a user-input device, a microphone and speaker 128 for audio input/output, and, optionally, a keyboard, mouse, or other user input device. Additionally, the user-interface hardware 108 may include a vibration motor 130 and/or one or more lights (e.g., light-emitting diodes (LED)) separate from the screen that can serve to alert the patient to the detection of a SAR episode. Like the computational facility 106, the user-interface hardware 108 may be distributed across multiple devices.

FIG. 2 depicts various example devices that may be used, individually or in combination, to implement the system 100 of FIG. 1, in accordance with various embodiments. A smartwatch 200 (e.g., a watch with computational processing and networking functionality) equipped with heartbeat sensors 102 (and, optionally, accelerometers 104) may serve to acquire heartbeat (and accelerometer) data and provide a physically perceptible (e.g., visual, audio, or mechanical/vibration) alert if a SAR event occurs and, optionally, allow the user to confirm or deny, or independently report, SAR episodes, e.g., via double-tap on a touch-sensitive face of the smartwatch 200. The smartwatch 200 may also provide all the computational functionality of the computational facility 106, thus serving as a stand-alone anxiety detection system 100.

Alternatively, to provide more processing power than the smartwatch 200 may have, the smartwatch 200 may send the data, e.g., via a Bluetooth connection, to a smartphone 202 or other portable computational device (e.g., tablet or personal digital assistant (PDA)) for processing. The smartphone 202 may, for instance, have a mobile app downloaded thereon that provides the processing functionality of the data preprocessing and featurization component 112 and the (e.g., trained) classification algorithm 110 and, optionally, allows for adjustments to the algorithm based on patient feedback. Detected SAR events may be communicated back from the smartphone 202 to the smartwatch 200, e.g., for activation of an alert or other mitigating action. Alternatively or additionally, the smartphone 202 may implement, e.g., via the downloaded mobile app, all or part of the mitigation component 114, which may tie into the existing phone/text and email applications serving as the communication component 116. Upon detection of a SAR event in the patient's heartbeat data, the mitigation component 114 may, for example, cause the smartphone 204 to display certain media content (e.g., related to stress-reducing techniques) on screen; prompt the user, e.g., via a pop-up display, to contact a healthcare provider or other trusted individual or call a hotline or emergency number (911); automatically send a text or audio message (e.g., via text or email) to one or more designated persons; open a social-networking site of a web browser to connect the patient to a self-help or support group or other social support network; and/or simply alert the user of the detected event, e.g., using conventional alarm functions (such as audio signals or vibration). As will be appreciated, the mitigation functionality may also be distributed between the smartphone 202 and smartwatch 200. Further, the detected events may be recorded in memory of the smartwatch 200, the smartphone 202, or both, e.g., in separate text files for the heartbeat and accelerometer data. User behavior tracked during the mitigating actions (e.g., making a call upon a prompt to do so, or progressing through a series of stress-reducing exercises) may also be stored for further analysis. Alternatively to communicating with a smartwatch 200, the smartphone 202 may receive the heartbeat data from separate sensors. Also, as noted above, corrections for patient movements may be made based on accelerometer data acquired by the built-in accelerometers of the smartphone 202.

In some embodiments, some or all of the data processing and classification to detect SAR events (e.g., as provided by components 110, 112) is performed by a remote computing system 204, e.g., a server computer or server farm. Such a remote server is generally configured for HIPPA (Health Insurance Portability and Accountability Act of 1996)-compliance to prevent misappropriation of patient data. The smartwatch 200 or smartphone 202 may send raw or preprocessed sensor data to the computing system 204, and the computing system 204 may return indications of any detected SAR events, via a telecommunications network 208 such as the internet or another wide-area network. At least a first link in the connection is typically wireless, consistent with the portability of the smartwatch 200 and smartphone 202. The smartwatch 200 and/or smartphone 202 may then perform one or more mitigating actions (e.g., as described above) locally to the patient. In some embodiments, the remote computing system may store records of SAR events and/or the data on which their detection is based as well as, optionally, information (including user behavioral data) pertaining to the mitigating actions; the data may, for instance, be stored in a central databank, and may be accessible by the patient's healthcare provider and/or aggregated across patient populations and anonymized for research and improvements to the classification algorithm.

In some embodiments, the smartwatch 200, smartphone 202, and remote computing system 204 may all cooperate to provide the overall functionality of the stress detection system 100. For example, sensor data acquired by the smartwatch 200 may be sent to the smartphone 202, which forwards it to the remote computing system for processing and takes mitigating actions in response to detected SAR events. As will be appreciated, other devices and combinations of devices may be used to implement the system 100. In general, at least one device is wearable (e.g., attachable to the patient's wrist, ankle, chest, other body part, or clothing) or portable (e.g., in the patient's pocket or handheld), allowing the patient to monitor his condition wherever he goes.

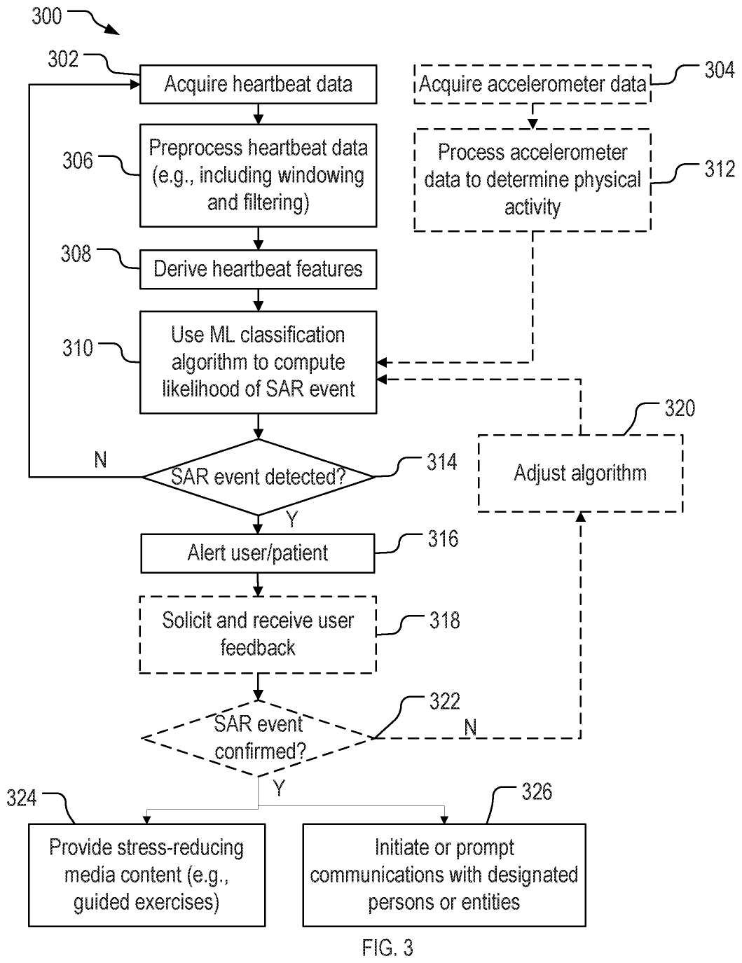

FIG. 3 is a flowchart illustrating a method of managing a SAR disorder with a system 100, in accordance with various embodiments. Assuming that a trained classification algorithm is available, the method 300 generally begins with the acquisition of time-series heartbeat data (act 302), and optionally time-series accelerometer data (act 304). Data acquisition is generally continuous, although conditions of the sensor, device, and/or patient may sometimes result in missing data values within the time series. In some embodiments, the (analog) heartbeat sensor signal is sampled at a rate sufficient to resolve the waveforms of individual heartbeats. In other embodiments, the signal is processed (e.g., by electronic circuitry internal to the sensors) to provide simply an average heart rate over a specified time window as sensor output. For example, the heart rate may be averaged over five-second intervals (corresponding to heart rate measurements at a frequency of 0.2 Hz). The heartbeat (e.g., heart rate) data is preprocessed (act 306) and/or featurized (act 308) to generate the input for a subsequently employed classification algorithm. In some embodiments, preprocessing the data involves windowing. For example, time-series heart rate data may be aggregated over one-minute sliding windows (each containing, e.g., 12 data samples for 0.2 Hz heart rate data) with 50% overlap between adjacent windows. Of course, other window sizes (e.g., a few minutes in length) and/or measurement rates may also be used. Further, Kalman filter imputation of missing values (a technique well-known to those of ordinary skill in the art) may be used to fill in gaps in the data, e.g., up to a cutoff for the number of consecutive missing values (e.g., a maximum of five missing values) beyond which the data imputation is deemed unreliable.

Features derived from the preprocessed heartbeat data (in act 308) may include statistical features (e.g., mean, variance, and quantiles or other distributional parameters computed for each time window), spectral features (e.g., Fourier and wavelet components), and/or non-linear or other features (e.g., sample entropy, time of last maximum value, frequency of minimum and maximum values, etc.). Among the large number of possible features, a smaller set of features to be computed may be selected based on associated high resulting classification performance. For instance, in some embodiments, the features that flow into the classification include six features representing Fourier components and one feature each representing an aggregated linear trend (e.g., linear least-squares regression for values of the time series that were aggregated over chunks versus the sequence from 0 up to 50), change quantile (e.g., average of consecutive changes of heart rate time series inside a corridor between quantiles 0.4 and 0.6 or some other pair of specified quantiles), and energy ratio (e.g., ratio of the sum of squares of a chunk of the time series to the sum of squares of the whole time series within the window).

The preprocessed (e.g., windowed and Kalman-filtered) time-series heartbeat data or time-series feature data derived therefrom is fed into a machine-learning algorithm to classify each time window as normal versus containing a SAR event (act 310). Suitable machine-learning algorithms and models are well-known to those of ordinary skill in the art and include, for example, support vector machines (SVMs), decision trees, random forests, neural networks, and convolutional neural networks (CNNs) or other deep neural networks. The type of input may depend on the particular algorithm. For example, SVM, decision tree, and random forest models may operate on heartrate features, whereas CNNs and neural networks may operate on the (Kalman-filtered) raw heartbeat data. In accordance with various embodiments, the machine-learning classification algorithm outputs, for each time window, the probability (likelihood) that a SAR event occurred during that window. With a specified threshold, this probability can be converted into a binary classification (i.e., normal versus SAR event). In accordance with various embodiments, the data pre-processing and classification happen in real time, allowing immediate action to be taken in the event the patient experiences a SAR episode.

In some embodiments, accelerometer data (acquired in act 304), e.g., sampled at 50 Hz, is used to assess the physical activity level of the user and take that information into account when determining whether a SAR event has occurred in a given time window. The accelerometer data may be processed (act 312) to determine and characterize physical activity, e.g., in terms of a computed velocity of the patient, a classification into one of multiple types of activity (e.g., running vs. bicycling, etc.), or implicitly via a set of features derived from the data. Accelerometer data processing may involve windowing, filtering, and/or featurizing as described above for heartbeat data. As shown, the processed accelerometer data may flow directly into the classification algorithm used to compute SAR-event probabilities, where it serves to discriminate between SAR events and heightened physical activity. Alternatively, detected SAR events may be post-processed to filter out seeming SAR events that are, in reality, due to increased activity; or heartbeat data processing may be suspended altogether if the physical activity is too high to allow for reliable SAR event detection. Alternatively or additionally to the use of accelerometer data, SAR-event detection may also take into account self-reported physical activity.

Upon detection of a SAR event (in act 314), the patient may be alerted of the occurrence of such event, e.g., by a visual, audible, or tactile (e.g., vibration) signal (act 316). In some embodiments, the system may solicit user input from the patient to give the patient the opportunity to confirm or deny that she, in fact, experiences a SAR episode (act 318). The user feedback, in particular any refutation of a SAR event, may be used to adjust and/or refine the classification algorithm (act 320), e.g., to reduce the number of false positives. The patient may also be allowed to independently report SAR events to the anxiety detection system to facilitate adjustments that increase the false negative rate.

If the SAR event is confirmed (in act 322) (or if the confirmation acts 318, 320 are skipped), the method 300 may proceed to one or more mitigating actions (beyond the alert). For example, the system 100 may provide or offer media content geared towards calming the patient (act 324). Such content may include, e.g., calming music/sounds and/or images/video, and/or instructions (whether provided in text, image, or audio form, or a combination thereof) for certain calming, stress-reducing breathing, muscle-relaxation, mindfulness, or other exercises. The system 100 may also automatically contact, or prompt the user to contact, one or more designated persons or entities, such as a medical professional (e.g., a physician, psychologist or psychiatrist, nurse, etc.) or other trusted individual (e.g., a close friend or relative), a group of people experiencing similar problems (e.g., a support group), or an organization or organizational entity involved in rendering aid to people in distress (e.g., 911 or a crisis hotline) (act 326).

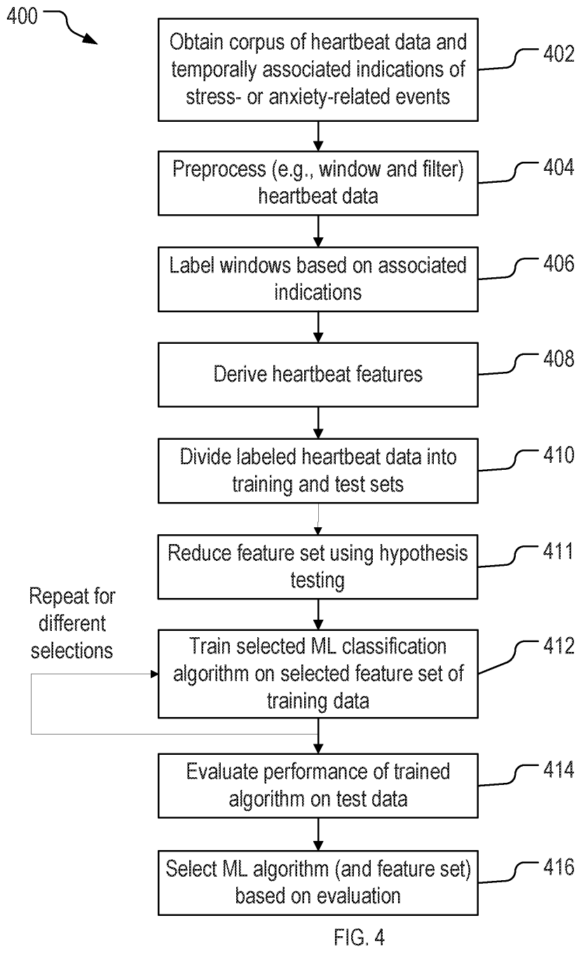

Having described how SAR events can be detected with machine-learning technology, a method for creating and training a suitable machine-learning algorithm is now described with reference to FIG. 4. The method 400 involves obtaining a corpus of data (act 402) on which machine-learning classification algorithms can be trained and tested. In general, such data includes continuous time-series heartbeat data acquired from one or more patients, along with temporally associated indications of SAR clinical events reported by the one or more patients. Patients may, for instance, be asked to wear heartbeat sensors (e.g., as integrated into smartwatches) continuously for a period of time, and to immediately report any SAR clinical events they perceive during that time period via a suitable user input, e.g., a double-tap or other touch based gesture performed on a touchscreen of the smartwatch (or other device); such self-reported SAR events are stored along with a time stamp. Optionally, the corpus of data may further include accelerometer data acquired from the patient(s) concurrently with the heartbeat data, and/or patient-reported indications of heightened physical activity. The heartbeat (and, optionally, accelerometer) data may be collected from a larger patient population (e.g., tens or hundreds of patients) to allow creating a patient-unspecific algorithm that captures statistical correlations between certain heartbeat signatures and SAR events. Once trained on the full corpus, the algorithm may be refined based on data for a particular patient. Alternatively, patient-specific data may be used from the outset to generate a custom algorithm. In any case, while the systems and methods described herein are generally applicable to any SAR disorder, the machine-learning algorithm can be custom-generated and trained for a particular psychiatric disorder or condition based on training data collected from patients with that particular condition. In some embodiments, the data is collected from people afflicted with PTSD. The reported SAR events for PTSD patients may be, specifically, (hyper-)arousal events. Other disorders that may benefit from monitoring and management with the approach described herein include, e.g., generalized anxiety disorder, panic disorder, and acute stress disorder.

The heartbeat data (and/or optional accelerometer data) may be preprocessed in the same manner as described with reference to FIG. 3 for real-time data processing during life anxiety detection (act 404). For instance, the heartbeat data may be windowed and Kalman-filtered. For training purposes, the windows are furthermore classified (or “labeled”) as “normal” or as including an SAR event (act 406), depending on whether a user-reported SAR event is associated with the respective window. The preprocessed data may be featurized (act 408). Since the best-performing features are generally not known a priori; a large number of features may initially be computed for subsequent testing. The labeled, featurized data is divided into training and test datasets, e.g., according to a 70% training/30% test split (act 410).

In some embodiments, a set of relevant features is identified among the large set of computed features, e.g., using the Benjamini Hochberg procedure (act 411). The Benjamini Hochberg procedure is a hypothesis-testing-based method that uses a null hypothesis that a feature has no predictive power and conducts a statistical test to attempt to reject the null hypothesis. Features for which the null-hypothesis is rejected according to a specified threshold of the p-value (e.g., p<0.05) are retained in the feature set subsequently used as input to the machine-learning algorithm. The Benjamini Hochberg procedure may be applied to the training dataset, withholding the test dataset to avoid bias in the final prediction result.

The training data may be used to train multiple machine-learning algorithms (e.g., as listed above, SVM, decision tree, random forest, neural network, CNN), and each algorithm may be trained on multiple features or feature sets to evaluate their respective performance (act 412). For any particular selection of algorithm and features, the training may be achieved with a suitable standard supervised learning algorithm. In general, supervised learning involves applying the selected machine-learning algorithm or model to the selected feature set to generate a predicted output (e.g., a predicted classification), which is then compared with the label (reflecting the ground truth). The difference between prediction and ground truth, or “cost,” serves as feedback to adjust parameters and/or hyperparameters of the model (e.g., weights in a neural network, maximum depth of a decision tree, cost and kernel of an SMV, etc.). Learning algorithms for optimizing the parameters are well-known to those of ordinary skill in the art and include, e.g., backpropagation of errors.

After the machine-learning algorithms have been optimized using the training data, their performance can be measured on the test data (act 414) by, generally speaking, quantifying the discrepancy between the predictions generated by the trained algorithm and the ground truth. Example performance metrics include the rates of false positives and false negatives, the F-measure (the weighted harmonic mean of the precision and recall of the classification), the area under the receiver operating characteristic (ROC) curve, and others. Based on the relative classification performance of various tested machine-learning algorithms and/or various feature sets, a high-performing combination may be selected for deployment (that is, for use in an anxiety detection system such as, e.g., system 100). In some embodiments, a reduced feature set (e.g., as determined by the Benjamini Hochberg procedure) achieves higher classification performance than a larger feature set.

Various software packages to perform some of the individual operations of methods 300, 400 are readily available commercially or can be created by a person of ordinary skill in the art without undue experimentation. For example, the “R” programming language provides an “ImputeTS” package for Kalman-filter imputation, a “Caret” package for fitting various machine-learning models to the data, and a “pROC” package for assessing statistical difference between ROC curves, and the “TSFRESH” package in Python facilitates comprehensive feature generation.

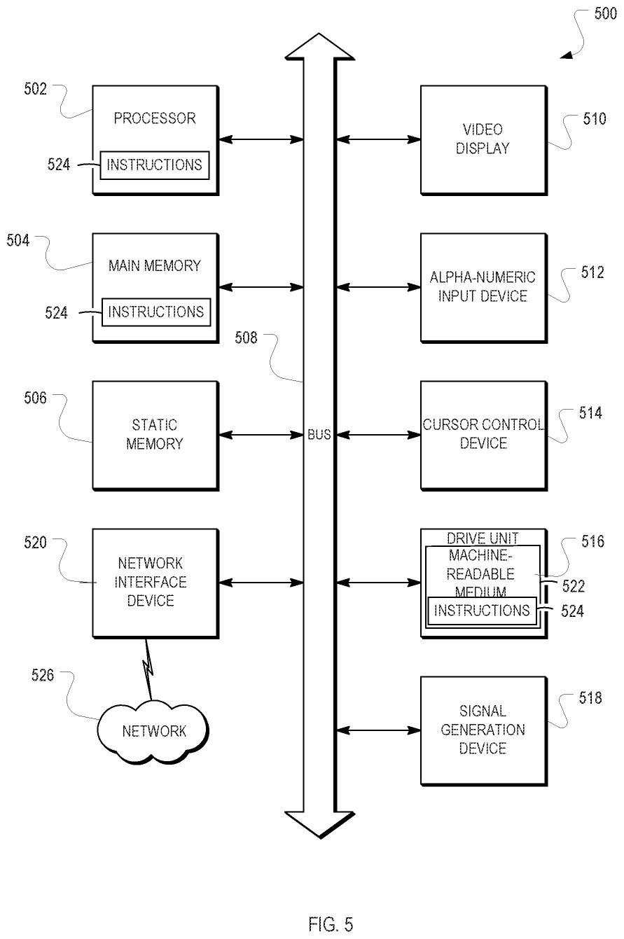

FIG. 5 is a schematic block diagram of an example computing system that may be used for performing anxiety detection and management functionality in accordance with various embodiments, e.g., implementing any one or more of the devices 200, 202, 204 of FIG. 2. The example computing system 500 takes the form of a machine within which instructions for causing the machine to perform various of the methodologies discussed herein may be executed. In alternative embodiments, the machine operates as a standalone device or may be connected (e.g., networked) to other machines. In a networked deployment, the machine may operate in the capacity of a server or a client machine in server-client network environment, or as a peer machine in a peer-to-peer (or distributed) network environment. The machine may be a personal computer (PC), a tablet PC, a set-top box (STB), a PDA, a cellular telephone, a web appliance, a network router, switch or bridge, or any machine capable of executing instructions (sequential or otherwise) that specify actions to be taken by that machine. Further, while only a single machine is illustrated, the term “machine” shall also be taken to include any collection of machines that individually or jointly execute a set (or multiple sets) of instructions to perform any one or more of the methodologies discussed herein.

The example computing system 500 includes a processor 502 (e.g., a central processing unit (CPU), a graphics processing unit (GPU) or both), a main memory 504 and a static memory 506, which communicate with each other via a bus 508. The computer system 500 may further include a video display unit 510 (e.g., a liquid crystal display (LCD) or a cathode ray tube (CRT)). The computer system 500 also includes an alphanumeric input device 512 (e.g., a keyboard), a cursor control device 514 (e.g., a mouse), a disk drive unit 516, a signal generation device 518 (e.g., a speaker) and a network interface device 520.

The disk drive unit 516 includes a machine-readable medium 522 on which are stored one or more sets of instructions and data structures (e.g., software) 524 embodying or used by any one or more of the methodologies or functions described herein. The instructions 524 may also reside, completely or at least partially, within the main memory 504, static memory 506, and/or within the processor 502 during execution thereof by the computer system 500, the main memory 504 and the processor 502 also constituting machine-readable media.

While the machine-readable medium 522 is shown in an example embodiment to be a single medium, the term “machine-readable medium” may include a single medium or multiple media (e.g., a centralized or distributed database, and/or associated caches and servers) that store the one or more instructions or data structures. The term “machine-readable medium” shall also be taken to include any tangible medium that is capable of storing, encoding or carrying instructions for execution by the machine and that cause the machine to perform any one or more of the methodologies of the present invention, or that is capable of storing or encoding data structures used by or associated with such instructions. The term “machine-readable medium” shall accordingly be taken to include, but not be limited to, solid-state memories, and optical and magnetic media. Specific examples of machine-readable media include non-volatile memory, including by way of example, semiconductor memory devices (e.g., Erasable Programmable Read-Only Memory (EPROM), Electrically Erasable Programmable Read-Only Memory (EEPROM)) and flash memory devices; magnetic disks such as internal hard disks and removable disks; magneto-optical disks; and CD-ROM and DVD-ROM disks. All such machine-readable storage media are hardware devices suitable for storing data and/or instructions for a suitable period of time to enable use by the machine, and are therefore non-transitory.

The instructions 524 may further be transmitted or received over a communications network 526 using a transmission medium. The instructions 524 may be transmitted using the network interface device 1320 and any one of a number of well-known transfer protocols (e.g., HTTP). Examples of communication networks include a LAN, a WAN, the Internet, mobile telephone networks, Plain Old Telephone (POTS) networks, and wireless data networks (e.g., WiFi and WiMax networks). The term “transmission medium” shall be taken to include any intangible medium that is capable of storing, encoding or carrying instructions for execution by the machine, and includes digital or analog communications signals or other intangible media to facilitate communication of such software.

Having described various aspects and features of the inventive subject matter, the following numbered examples are provided as illustrative embodiments:

1. A method for managing a stress- or anxiety-related (SAR) disorder in a patient, the method comprising: acquiring time-series heartbeat data from the patient using one or more wearable heartbeat sensors; processing the heartbeat data in real time, using a machine-learning classification algorithm to detect a SAR clinical event in the acquired heartbeat data, the machine-learning classification algorithm trained on heartbeat data for one or more patients having a SAR disorder in conjunction with temporally associated indications of SAR clinical events reported by the one or more patients; and performing a mitigating action in response to detecting the SAR clinical event.

2. The method of example 1, further comprising storing the detected SAR clinical event in memory in association with a timestamp.

3. The method of example 1 or example 2, further comprising, contemporaneously with acquiring the heartbeat data, acquiring time-series accelerometer data indicative of movements of the patient using one or more wearable accelerometers, wherein the heartbeat data is processed in conjunction with the accelerometer data, the machine-learning classification algorithm being trained to discriminate between heartbeat signatures associated with SAR clinical events and heartbeat signatures associated with high physical activity.

4. The method of any one of examples 1-3, further comprising receiving a contemporaneous report of a SAR clinical event from the patient and adjusting the machine-learning classification algorithm based thereon.

5. The method of any one of examples 1-4, wherein processing the heartbeat data comprises extracting time-series feature sets from the heartbeat data and classifying the feature sets to generate time-series SAR event likelihood output.

6. The method of any one of examples 1-5, wherein the SAR disorder is Post-Traumatic Stress Disorder (PTSD) and the SAR event is a hyperarousal event.

7. The method of any one of examples 1-6, wherein the mitigating action comprises activating a physical alert.

8. The method of any one of examples 1-7, wherein the mitigating action comprises automatically communicating the SAR clinical event to a contact designated by the patient.

9. The method of any one of examples 1-8, wherein the mitigating action comprises prompting the patient to initiate electronic communications with a designated contact person.

10. The method of any one of examples 1-9, wherein the mitigating action comprises providing, via a user interface of a portable device, electronic content to guide a user through one or more stress-reducing exercises.

11. The method of any one of examples 1-10, wherein the one or more stress-reducing exercises comprise at least one of breathing techniques, active muscle relaxation techniques, or mindfulness techniques.

12. A system for managing a stress- or anxiety-related (SAR) disorder in a patient, the system comprising one or more portable devices that include: one or more wearable heartbeat sensors to acquire time-series heartbeat data from a patient; a computational facility to process the heartbeat data in real time, using a machine-learning classification algorithm to detect a SAR clinical event in the acquired time-series heartbeat data, the machine-learning classification algorithm trained on time-series heartbeat data for one or more patients having a SAR disorder in conjunction with temporally associated indications of SAR clinical events reported by the one or more patients; and a user interface to perform a mitigating action in response to detecting the SAR clinical event.

13. The system of example 12, wherein the one or more wearable heartbeat sensors, the computational facility, and the user interface are integrated into a single wearable device.

14. The system of example 13, wherein the wearable device is a smartwatch.

15. The system of example 12, wherein the one or more wearable heartbeat sensors are integrated into a wearable monitor device that is communicatively couplable to a mobile communication device comprising the computational facility and the user interface.

16. The system of example 15, wherein the mobile communication device is a smartphone.

17. The system of any one of examples 12-16, further comprising one or more wearable accelerometers to acquire time-series accelerometer data indicative of movements of the patient, wherein the computational facility is to process the heartbeat data in conjunction with the accelerometer data, the machine-learning classification algorithm being trained to discriminate between heartbeat signatures associated with SAR clinical events and heartbeat signatures associated with high physical activity.

18. The system of any one of examples 12-17, wherein the user interface comprises a touchscreen and is configured to record a patient-reported SAR event upon a touch gesture performed on the touchscreen.

19. A system for managing a stress- or anxiety-related (SAR) disorder in a patient, the system comprising: one or more portable devices that include: one or more wearable heartbeat sensors to acquire time-series heartbeat data from a patient; a network interface to send the acquired heartbeat data in real time to a computational facility for real-time processing and to receive a signal indicative of a SAR clinical event detected in the time-series heartbeat data from the processing facility in real time; and a user interface to perform a mitigating action in response to the signal indicative of the SAR clinical event.

20. The system of example 19, further comprising the processing facility, wherein the computational facility is configured to use a machine-learning classification algorithm to detect the SAR clinical event in the acquired time-series heartbeat data, the machine-learning classification algorithm trained on time-series heartbeat data for one or more patients having a SAR disorder in conjunction with temporally associated indications of SAR clinical events reported by the one or more patients.

21. One or more computer-readable media storing instructions for execution by one or more processors of a machine, the instructions, when executed, causing the one or more processors to perform operations comprising: in response to receipt of time-series heartbeat data acquired from a patient, processing the heartbeat data in real time, using a machine-learning classification algorithm to detect a SAR clinical event in the acquired time-series heartbeat data, the machine-learning classification algorithm trained on time-series heartbeat data for one or more patients having a SAR disorder in conjunction with temporally associated indications of SAR clinical events reported by the one or more patients; and generating an output indicative of the detected SAR clinical event.

Although embodiments have been described with reference to specific example embodiments, it will be evident that various modifications and changes may be made to these embodiments without departing from the broader scope of the invention. Accordingly, the specification and drawings are to be regarded in an illustrative rather than a restrictive sense. The accompanying drawings that form a part hereof show by way of illustration, and not of limitation, specific embodiments in which the subject matter may be practiced. The embodiments illustrated are described in sufficient detail to enable those skilled in the art to practice the teachings disclosed herein. Other embodiments may be used and derived therefrom, such that structural and logical substitutions and changes may be made without departing from the scope of this disclosure. This description, therefore, is not to be taken in a limiting sense, and the scope of various embodiments is defined only by the appended claims, along with the full range of equivalents to which such claims are entitled.

What is claimed is:

1. A method for managing a stress- or anxiety-related (SAR) disorder in a patient, the method comprising:
acquiring time-series heartbeat data from the patient using one or more wearable heartbeat sensors;
processing the heartbeat data in real time, using a machine-learning classification algorithm to detect a SAR clinical event in the acquired heartbeat data, the machine-learning classification algorithm trained on heartbeat data for one or more patients having a SAR disorder in conjunction with temporally associated indications of SAR clinical events reported by the one or more patients; and
performing a mitigating action in response to detecting the SAR clinical event.

2. The method of claim 1, further comprising storing the detected SAR clinical event in memory in association with a timestamp.

3. The method of claim 1, further comprising, contemporaneously with acquiring the heartbeat data, acquiring time-series accelerometer data indicative of movements of the patient using one or more wearable accelerometers, wherein the heartbeat data is processed in conjunction with the accelerometer data, the machine-learning classification algorithm being trained to discriminate between heartbeat signatures associated with SAR clinical events and heartbeat signatures associated with high physical activity.

4. The method of claim 1, further comprising receiving a contemporaneous report of a SAR clinical event from the patient and adjusting the machine-learning classification algorithm based thereon.

5. The method of claim 1, wherein processing the heartbeat data comprises extracting time-series feature sets from the heartbeat data and classifying the feature sets to generate time-series SAR event likelihood output.

6. The method of claim 1, wherein the SAR disorder is Post-Traumatic Stress Disorder (PTSD) and the SAR event is a hyperarousal event.

7. The method of claim 1, wherein the mitigating action comprises activating a physical alert.

8. The method of claim 1, wherein the mitigating action comprises automatically communicating the SAR clinical event to a contact designated by the patient.

9. The method of claim 1, wherein the mitigating action comprises prompting the patient to initiate electronic communications with a designated contact person.

10. The method of claim 1, wherein the mitigating action comprises providing, via a user interface of a portable device, electronic content to guide a user through one or more stress-reducing exercises.

11. The method of claim 10, wherein the one or more stress-reducing exercises comprise at least one of breathing techniques, active muscle relaxation techniques, or mindfulness techniques.

12. A system for managing a stress- or anxiety-related (SAR) disorder in a patient, the system comprising one or more portable devices that include:
one or more wearable heartbeat sensors to acquire time-series heartbeat data from a patient;
a computational facility to process the heartbeat data in real time, using a machine-learning classification algorithm to detect a SAR clinical event in the acquired time-series heartbeat data, the machine-learning classification algorithm trained on time-series heartbeat data for one or more patients having a SAR disorder in conjunction with temporally associated indications of SAR clinical events reported by the one or more patients; and
a user interface to perform a mitigating action in response to detecting the SAR clinical event.

13. The system of claim 12, wherein the one or more wearable heartbeat sensors, the computational facility, and the user interface are integrated into a single wearable device.

14. The system of claim 13, wherein the wearable device is a smartwatch.

15. The system of claim 12, wherein the one or more wearable heartbeat sensors are integrated into a wearable monitor device that is communicatively couplable to a mobile communication device comprising the computational facility and the user interface.

16. The system of claim 15, wherein the mobile communication device is a smartphone.

17. The system of claim 12, further comprising one or more wearable accelerometers to acquire time-series accelerometer data indicative of movements of the patient, wherein the computational facility is to process the heartbeat data in conjunction with the accelerometer data, the machine-learning classification algorithm being trained to discriminate between heartbeat signatures associated with SAR clinical events and heartbeat signatures associated with high physical activity.

18. The system of claim 12, wherein the user interface comprises a touchscreen and is configured to record a patient-reported SAR event upon a touch gesture performed on the touchscreen.

19. A system for managing a stress- or anxiety-related (SAR) disorder in a patient, the system comprising:
one or more portable devices that include:
one or more wearable heartbeat sensors to acquire time-series heartbeat data from a patient;
a network interface to send the acquired heartbeat data in real time to a computational facility for real-time processing and to receive a signal indicative of a SAR clinical event detected in the time-series heartbeat data from the processing facility in real time; and
a user interface to perform a mitigating action in response to the signal indicative of the SAR clinical event.

20. The system of claim 19, further comprising the processing facility, wherein the computational facility is configured to use a machine-learning classification algorithm to detect the SAR clinical event in the acquired time-series heartbeat data, the machine-learning classification algorithm trained on time-series heartbeat data for one or more patients having a SAR disorder in conjunction with temporally associated indications of SAR clinical events reported by the one or more patients.

21. One or more computer-readable media storing instructions for execution by one or more processors of a machine, the instructions, when executed, causing the one or more processors to perform operations comprising:
in response to receipt of time-series heartbeat data acquired from a patient, processing the heartbeat data in real time, using a machine-learning classification algorithm to detect a SAR clinical event in the acquired time-series heartbeat data, the machine-learning classification algorithm trained on time-series heartbeat data for one or more patients having a SAR disorder in conjunction with temporally associated indications of SAR clinical events reported by the one or more patients; and
generating an output indicative of the detected SAR clinical event.
